(19) United States
(12) Patent Application Publication
(10) Pub. No.: US 2020/0265951 A1
(43) Pub. Date: Aug. 20, 2020

(54) VIRTUAL INFUSION PUMPS

(71) Applicant: KONINKLIJKE PHILIPS N.V., EINDHOVEN (N;)

(72) Inventors: John CRONIN, BONITA SPRINGS, FL (US); Steven PHILBIN, BONITA SPRINGS, FL (US)

(21) Appl. No.: 16/063,715

(22) PCT Filed: Dec. 22, 2016
(86) PCT No.: PCT/IB2016/057912

§ 371 Date: Jun. 19, 2018

Related U.S. Application Data
(60) Provisional application No. 62/274,068, filed on Dec. 31, 2015.

(51) Int. Cl. G16H 40/67; G16H 20/17; G16H 70/20; G16H 50/20; G16H 10/60; G16H 40/20; G06F 16/953; G05B 13/02; G06N 5/02; G06N 5/04; A61M 5/172; H04L 12/58

(52) U.S. Cl. CPC G16H 40/67; G16H 20/17; G16H 70/20; G16H 50/20; G16H 10/60; G16H 40/20; G16H 80/00; G05B 13/028; G06N 5/02; G06N 5/04; A61M 5/1723; H04L 51/04; G06F 16/953

(57) ABSTRACT

Systems and methods for providing a virtual infusion pump 120 are provided. An infusion pump may be used in medical treatment of a patient. One or more sensors may monitor one or more medical factors relevant to a patient. Such monitored factors may be identified as being associated with a recommended action. A virtual infusion pump 120 controller may then formulate instructions for executing the recommended action, wherein the instructions are sent to the infusion pump for execution.

### CROSS-REFERENCE TO RELATED APPLICATIONS

The present application claims the priority benefit of U.S. provisional application No. 62/274,068 filed Dec. 31, 2015 and entitled “Virtual Infusion Pumps,” the disclosure of which is incorporated herein by reference in its entirety.

### BACKGROUND OF THE INVENTION

### Field of the Invention

The present invention generally relates to infusion pumps. More specifically, the present invention relates to virtual infusion pumps.

### Description of the Related Art

Infusion pumps are devices that deliver fluids, nutrients, and/or medication directly into a patient's body in controlled amounts and at controlled rates. Infusion pumps may deliver intravenous, subcutaneous, arterial, or epidural infusions. Presently available infusion systems and pumps currently do not have the ability to intelligently use electronic medical records to adjust treatment in real-time.

By contrast, presently available “smart” infusion pumps may be complicated, unwieldy, and/or quite expensive. Moreover, many medical facilities may already have one or many infusion pump units that are still operable and functional, despite lacking intelligent decision-making abilities.

Magnetic resonance imaging (MRI) is a procedure generally used to create images of internal organs and structures in the human body by using magnetic fields. Patients undergoing an MRI may need to continue infusion treatments that are administered by one or more MRI-compatible infusion pumps.

There is, therefore, a need in the art for improved systems and methods for providing intelligent, data-driven virtual infusion pumps.

### SUMMARY OF THE CLAIMED INVENTION

Embodiments of the present invention include systems and methods for providing a virtual infusion pump. An infusion pump may be used in medical treatment of a patient. One or more sensors may monitor one or more medical factors relevant to a patient. Such monitored factors may be identified as being associated with a recommended action. A virtual infusion pump controller may then formulate instructions for executing the recommended action, wherein the instructions are sent to the infusion pump for execution.

### BRIEF DESCRIPTION OF THE DRAWINGS

FIG. 1 illustrates an exemplary network environment in which a system for providing a virtual infusion pump may be implemented.

FIG. 2 is a flowchart illustrating an exemplary method for providing a virtual infusion pump.

FIG. 3 is a flowchart illustrating an exemplary method for controlling an infusion pump in accordance with a virtual infusion pump system.

FIG. 4 illustrates an exemplary virtual infusion pump recommendation graphic user interface (GUI).

FIG. 5 illustrates an exemplary infusion pump that may be used in a virtual infusion pump system.

FIGS. 6A-6B are a block diagram of an embodiment of a base GUI generated by the virtual infusion pump and a flowchart for a method performed by the execution of a base software according to some embodiments.

FIGS. 7A-7B are a block diagram of an embodiment of a chat room GUI generated by the virtual infusion pump and a flowchart for a method performed by the execution of a chat room software according to some embodiments.

FIGS. 8A-8B are a block diagram of an embodiment of a manuals GUI generated by the virtual infusion pump and a flowchart for a method performed by the execution of a manuals software according to some embodiments.

FIGS. 9A-9B are a block diagram of an embodiment of a safety GUI generated by the virtual infusion pump and a flowchart for a method performed by the execution of a safety software according to some embodiments.

FIGS. 10A-10B are a block diagram of an embodiment of a calculator GUI generated by the virtual infusion pump and a flowchart for a method performed by the execution of a calculator software according to some embodiments.

FIGS. 11A-11B are a block diagram of an embodiment of an other sensor GUI generated by the virtual infusion pump and a flowchart for a method performed by the execution of an other software according to some embodiments.

FIG. 12 illustrates an exemplary computing system that may be used to implement an embodiment of the present invention.

### DETAILED DESCRIPTION

Embodiments of the present invention include systems and methods for providing a virtual infusion pump. Embodiments of the present invention include systems and methods for providing a virtual infusion pump. An infusion pump may be used in medical treatment of a patient. One or more sensors may monitor one or more medical factors relevant to a patient. Such monitored factors may be identified as being associated with a recommended action. A virtual infusion pump controller may then formulate instructions for executing the recommended action, wherein the instructions are sent to the infusion pump for execution.

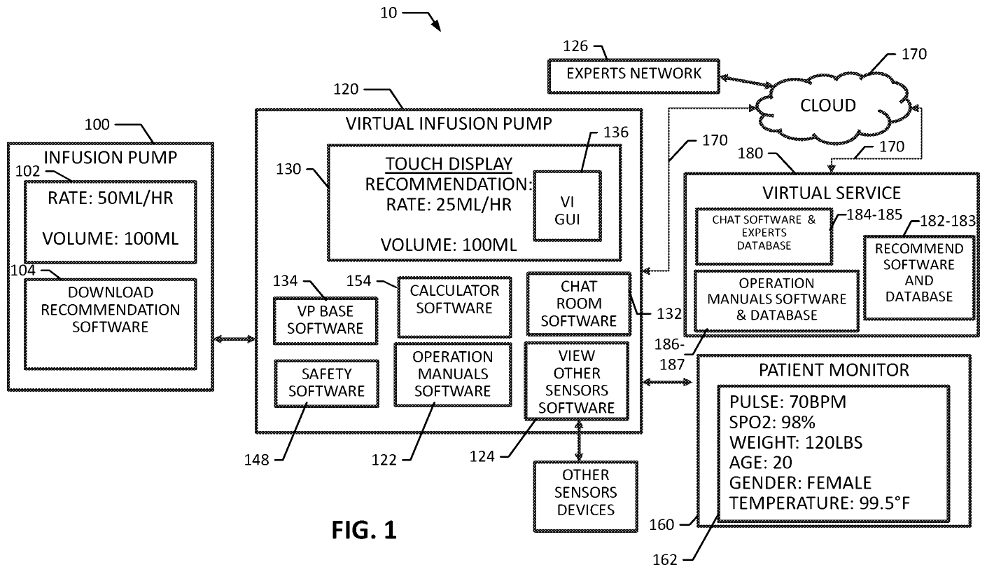

FIG. 1 illustrates an exemplary network environment 10 in which a system for providing a virtual infusion pump may be implemented. Such a network environment 10 may include a standard infusion pump 100 (as used herein a standard infusing pump is an infusing pump that lacks processing abilities, a virtual infusion pump 120, a patient monitor 160, cloud communication networks 170, and one or more cloud-based servers 180 that provides various services. Such devices may communicate with each other via hard-wired or wireless communications systems and networks known in the art.

The standard infusion pump 100 may include a display 102 that indicates such measurements as rate and volume of infusion, as well as the ability to communicate with virtual infusion pump 120 to download instructions therefrom. The standard infusion pump 100 is therefore capable of communicating with another device (e.g., virtual infusion pump 120 or other type of controller) to receive instructions to be execute by the infusion pump. The infusion pump may be associated with an input device 104 (e.g., button) that allows for recommendations to be downloaded on-demand or otherwise received in real-time.

The virtual infusion pump 120 is able to communicate with various different devices in the network environment 10 to obtain data, as well as to control the infusion pump. In some instances, the virtual infusion pump 120 may include manuals for a variety of different types of infusion pumps, including electronic manuals and instructions for maintenance and operations. Such manuals may be used in the formulation of instructions to a particular infusion pump of interest. Such instructions may pertain to adjusting operations of the infusion pump 100, including at least adjusting a flow rate or volume of infusion to a patient. In addition, virtual infusion pump 120 may include sensor software 124 or other executable instructions to access various sensors, as well as third party sources 126 including forums, chat rooms, and other expert database that may be useful for managing and controlling the infusion pump.

The virtual infusion pump 120 may be any number of different controllers or electronic user devices, such as general purpose computers, mobile phones, smartphones, personal digital assistants (PDAs), portable computing devices (e.g., laptop, netbook, tablets), desktop computing devices, handheld computing device, or any other type of computing device having a processor that is capable of communicating over wired and/or wireless communication networks. The virtual infusion pump 120 may also be configured to access data from other storage media, such as memory cards or disk drives as may be appropriate in the case of downloaded services. The virtual infusion pump 120 may include standard hardware computing components such as network and media interfaces, non-transitory computer-readable storage (memory), and processors for executing instructions that may be stored in memory.

Virtual infusion pump 120 may include or have access to a recommendations database 183 storing one or more recommendations for infusion pump operation (or operation adjustment) based on various factors. Such factors may include current information regarding the patient, including age, gender, weight, arterial oxygen saturation (SpO₂), and temperature. The recommendations may be based on rules associating certain combinations of patient data with one or more actions.

As such, virtual infusion pump 120 may evaluate such factors (e.g., as detected by various sensors or accessed from various databases) and based on such evaluation, formulate one or more recommendations as to how the infusion pump should operate. Where the recommendations may be formulated remotely (e.g., by cloud-based service such as those hosted at a cloud-based server 180), the virtual infusion pump 120 may provide data regarding factors relevant to the patient, download a responsive recommendation, and then provide instructions to the infusion pump based on the recommendation.

In some embodiments, virtual infusion pump 120 may evaluate various use cases or scenarios based on a given set of factors. Such evaluations may include one or more predicted outcomes based on one or more different actions that can be taken. Such predictions may be displayed on a screen or display 130, and the virtual infusion pump 120 may wait for approval or a selection before providing instructions to the infusion pump. In some embodiments, the virtual infusion pump 120 may further determine a best time to provide the instructions to the infusion pump and provide the instructions at the determined time rather than immediately sending the instructions.

The factors relied upon by the virtual infusion pump 120 may also be sensed, inputted, and/or displayed via a patient monitor 160 unit. Patient monitor 160 unit may therefore include or be associated with any type of sensor, medical measurement tool, display screen 162, touchscreen, or other input-output device (e.g., touchscreen, keyboard, keypad, other buttons, microphone, camera, and other peripheral devices) known in the art.

The c loud communication network 170 may include a local, proprietary network (e.g., an intranet) and/or may be a part of a larger wide-area network. The communication network 170 may be a local area network (LAN), which may be communicatively coupled to a wide area network (WAN) such as the Internet. The Internet is a broad network of interconnected computers and servers allowing for the transmission and exchange of Internet Protocol (IP) data between users connected through a network service provider. Examples of network service providers are the public switched telephone network, a cable service provider, a provider of digital subscriber line (DSL) services, or a satellite service provider. Communication networks 170 allow for communication between the various components of the network environment 10.

The cloud-based server(s) 180 that provide cloud services may include any type of server or other computing device as is known in the art, including standard hardware computing components such as network and media interfaces, non-transitory computer-readable storage (memory), and processors for executing instructions or accessing information that may be stored in memory. The functionalities of multiple servers may be integrated into a single server. Any of the aforementioned servers (or an integrated server) may take on certain client-side, cache, or proxy server characteristics. These characteristics may depend on the particular network placement of the server or certain configurations of the server.

In various aspects, the cloud-based server(s) 180 include various databases and execute various software applications that may provide data, such as instructions and recommendations to the virtual infusion pump 120. For example, the cloud-based server 180 may include a recommendations software and an associated database, generally indicated as 182-183, chat software and an associated experts database, generally indicated as 184-185, as well as operation manuals software and databases, generally indicated as 186-187 related to the various standard infusion pumps 100.

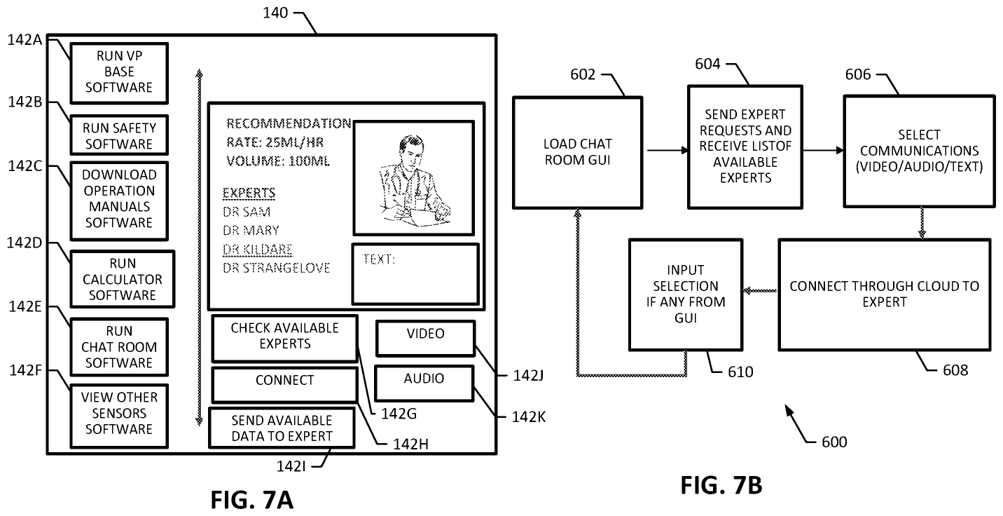

In one embodiment, the chat software 184 is initiated when the virtual infusion pump 120 loads and executes the chat room software 132, described and shown more fully below at FIG. 7. The chat software 184 sends data regarding the available experts in the experts database 185 to the user of the virtual infusion pump 120. When the user selects an expert and the type of communication desired, the chat software 184 generates the desired communication and transmits it along with any data from the virtual infusion pump 120 to the expert network 126.

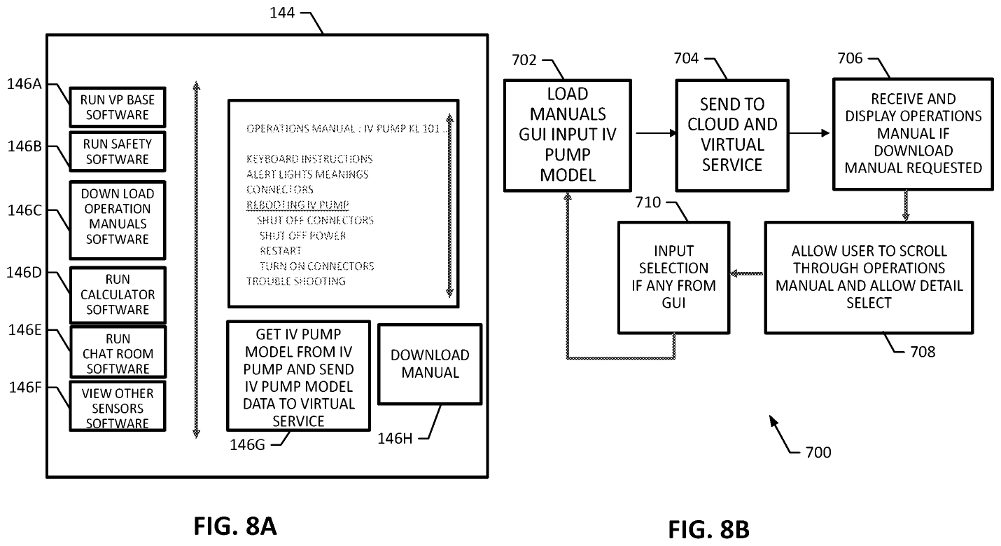

Similarly, the operations manuals software 186 provides data in response to a request from the virtual infusion pump's manual software 122, described and shown more fully below at FIG. 8. In one embodiment, the request includes the model number for the standard infusion pump 100 and the operations manuals software 186 identified the model of the standard pump in the database 187 and sends the available data back to the Virtual infusion pump 120.

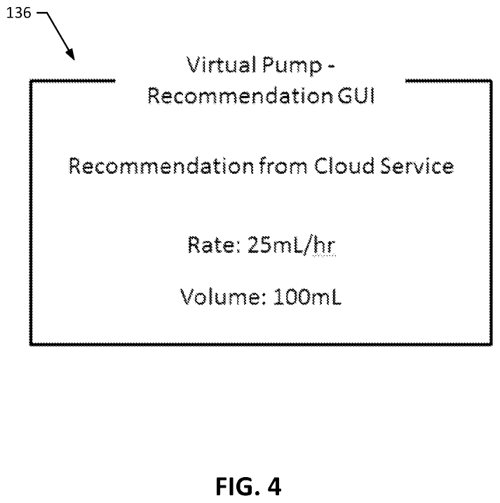

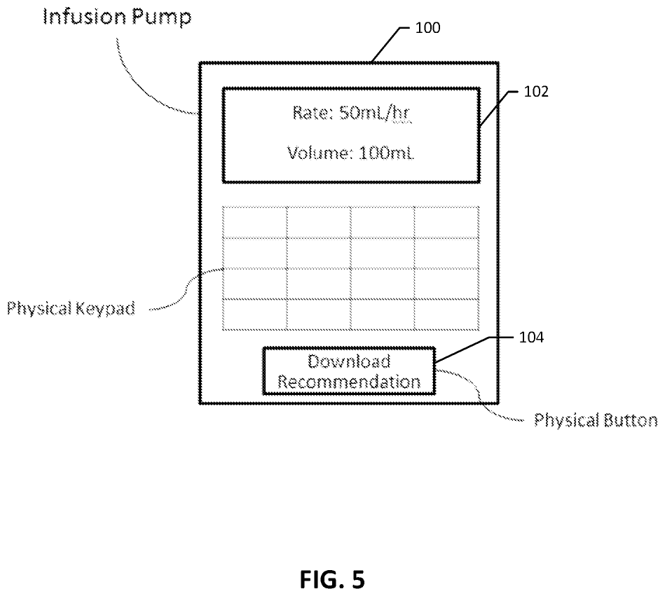

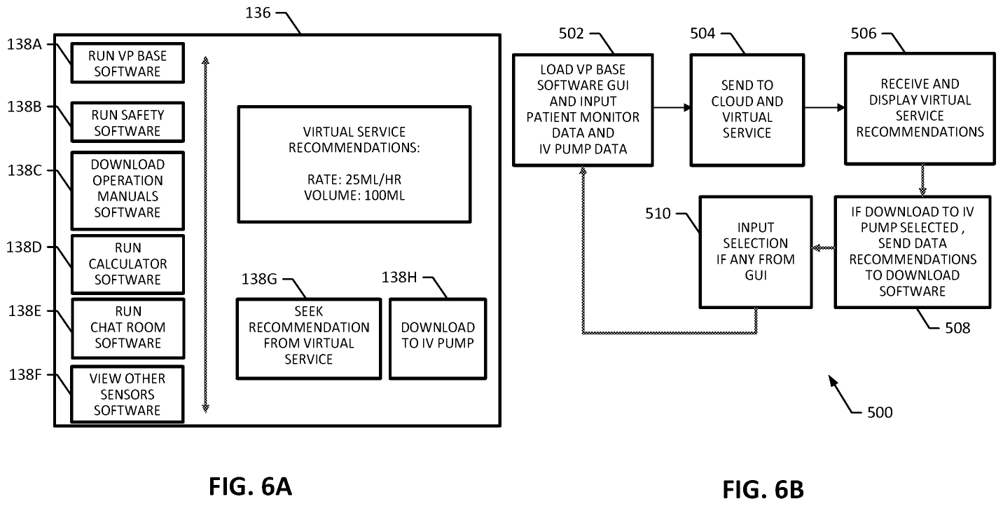

The recommend software 182 provides parameter and operational recommendations in response to a request from the virtual infusion pump's base software 134, described and shown more fully below at FIG. 6. In one embodiment, when the virtual infusion pump's base software 134 is executed and recommendations are requested, the base software sends all available data from the patient monitor 160 and the infusion pump 100 to the server 180. In response the recommend software 182 uses an Artificial intelligence (AI) engine, a rules engine, an expert systems engine, or combinations thereof to compare the data sent by the base software 134 against recommendations stored in the database 187. The recommend software 182 sends the best recommendation, for example desired pump settings. In another example, the recommendation may account for a patient who has high arterial oxygen saturation (SpO₂) data, and include a recommendation that the infusion pump flow should be lower than standard flow, as shown in FIGS. 4 and 5.

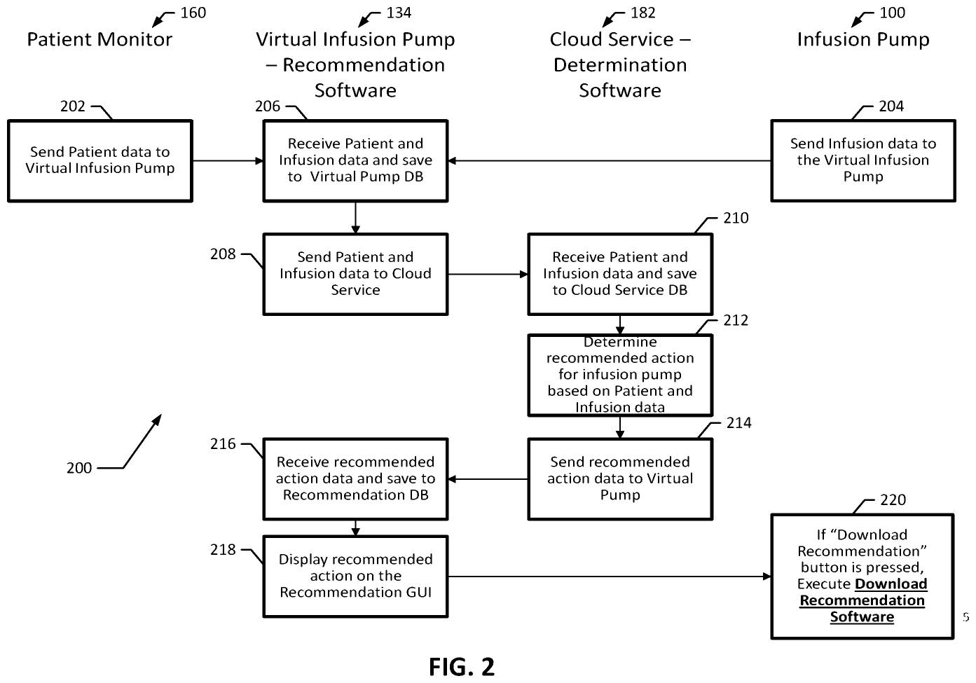

FIG. 2 is a flowchart illustrating an exemplary method 200 for providing a virtual infusion pump 120. The method 200 may be embodied as executable instructions in a non-transitory computer readable storage medium including but not limited to a CD, DVD, or non-volatile memory such as a hard drive. The instructions of the storage medium may be executed by a processor (or processors) to cause various hardware components of a computing device hosting or otherwise accessing the storage medium to effectuate the method. The steps 202-220 identified in FIG. 2 (and the order thereof) are exemplary and may include various alternatives, equivalents, or derivations thereof including but not limited to the order of execution of the same.

In the method of FIG. 2, the patient monitor 160 unit may send current patient data to the virtual infusion pump 120 at step 202, which may also receive current infusion data from an infusion pump currently in use by the patient at step 204. The virtual infusion pump 120 may then save such patient and infusion data to a virtual infusion pump 120 database at step 206. Such patient and infusion data may then be sent to a cloud-based server 180 for analysis at step 208. In alternative embodiments, the patient and infusion data may be analyzed locally at the virtual infusion pump 120.

Where the patient and infusion data is sent to the cloud-based server 180, the cloud-based server 180 may save the patient and infusion data in a cloud-based server 180 database at step 210 and then determine a recommended action for infusion pump to take based on such patient and infusion data at step 212. As noted above, such recommendation may be based on a database of recommendations correlating one or more actions to various sets of factors relevant to the patient.

Such recommendation may be sent to the virtual infusion pump 120 at step 214, which may save such recommendation in a recommendations database at step 216. In some instances, the virtual infusion pump 120 may further display the recommended action via a recommendation graphic user interface (GUI) on an associated display screen at step 218. The virtual infusion pump 120 may then generate control instructions for execution by the infusion pump in accordance with the recommendation. The control instructions may then be sent by the virtual infusion pump 120 to the infusion pump, if the infusion pump is set up to download recommendations from the virtual infusion pump 120. Upon receiving the instructions, the infusion pump may execute such instructions to perform a particular action in accordance with the recommendation at step 220.

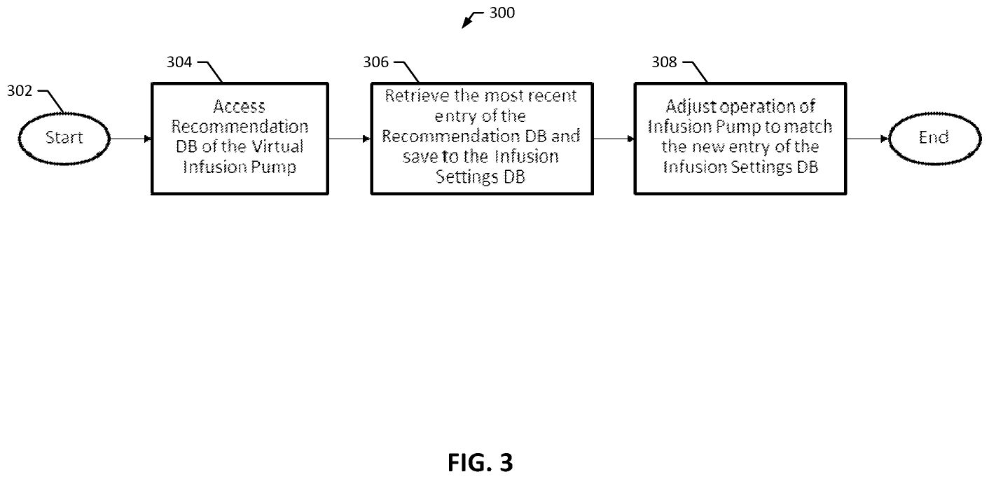

FIG. 3 is a flowchart illustrating an exemplary method 300 for controlling an infusion pump in accordance with a virtual infusion pump 120 system. The infusion pump may be prompted (e.g., via a “download recommendation” button) at step 302 to execute download recommendation software and to download recommendations from the virtual infusion pump 120. As such, the infusion pump may request or access a recommendation from the recommendation database at the virtual infusion pump 120 at step 304. Such recommendation (e.g., a most recent recommendation entry) may further be copied and saved to an infusion pump settings database at the infusion pump) at step 306. The infusion pump may then use the recommendation stored in the infusion pump settings database to adjust its operations (e.g., control of flow rate and volume of infusion) at step 308.

FIG. 4 illustrates an exemplary virtual infusion pump 120 recommendation graphic user interface (GUI). The virtual pump recommendation GUI 136 is displayed on a display screen 130 incorporated in or associated with the virtual infusion pump 120. The virtual pump recommendation GUI 136 displays the recommended action data received from the cloud server 180. The recommended action could include, for example, adjusting the flow rate and/or volume of infusion.

FIG. 5 illustrates an exemplary infusion pump 100 that may be used in a virtual infusion pump 120 system. Such infusion pump may include a screen that displays infusion data (e.g., flow rate and volume of infusion), a keypad, and a button to initiate downloading of one or more recommendations.

FIGS. 6A-6B are a block diagram of an embodiment of a base GUI 136 generated by the virtual infusion pump 120 and a flowchart for a method performed by the execution of the base software 134, respectively. In one embodiment, the base GUI 136 is generated by the virtual pump 120 for initiating execution of the base software 134, along with other software executable at the virtual pump. As shown, the base GUI 136 may also display any recommendations received at the virtual pump 120 and includes one or more other interactive input prompts 138A-H to initiate the execution of various actions or software.

FIG. 6B is a flowchart of a method 500 performed or caused to be performed by execution of the virtual pump base software 134 at a processor. In one aspect, the base software 134 continually loads data from the patient monitor 160 and the infusion pump 100 and continually sends it to the cloud-based resources to request any recommendations, if any. As shown, at step 502, the base GUI 136 is generated and data from the patient monitor and the infusion pump is received. At step 504, a request for recommendations that includes the data is sent to the cloud-based server 180. In response, a recommendation returned by the cloud-based server 180 is received and displayed at step 506. If an input to download the recommendations is made the infusion pump 100, then the recommendations are transmitted to the infusion pump at step 508. At step 510, inputs for additional actions, if any, are received and the method is repeated as desired.

FIGS. 7A-7B are a block diagram of an embodiment of a chat room GUI 140 generated by the virtual infusion pump 120 and a flowchart for a method performed by the execution of the chat room software 132, respectively. In one embodiment, the chat room GUI 140 is generated by the virtual pump 120 for initiating execution of the chat room software 132, along with other software executable at the virtual pump. As shown, the chat room GUI 140 may also display any recommendations received at the virtual pump 120 and includes one or more other interactive input prompts 142A-K to initiate the execution of various actions or software. As shown, the chat room GUI 140 allows the user to select and consult with an available doctor or other expert. The user can also communicate using audio, video, text, or combinations thereof. In various embodiments, all of the available data received at the virtual pump 120 may be sent to the expert.

FIG. 7B is a flowchart of a method 600 performed or caused to be performed by execution of the virtual pump chat room software 132 at a processor. As shown, at step 602, the chat room GUI 140 is generated. At step 604, a request for contacting an expert is received at the chat room GUI 140 and the request and support data, as desired, is sent to the cloud-based server 180, where an indication of the available experts is returned by the cloud-based server. At step 606, a user indicated the desired method of communication, while at step 608, communication with the selected expert is made via the cloud-based server 180. Any supporting data may also be sent to the expert at step 608. At step 610, inputs for additional actions, if any, are received and the method is repeated as desired.

FIGS. 8A-8B are a block diagram of an embodiment of a manuals GUI 144 generated by the virtual infusion pump 120 and a flowchart for a method performed by the execution of the manuals software 122, respectively. In one embodiment, the manuals GUI 144 is generated by the virtual pump 120 for initiating execution of the manuals software 122, along with other software executable at the virtual pump. As shown, the manuals GUI 144 may also display any manual data received at the virtual pump 120 and includes one or more other interactive input prompts 146A-H to initiate the execution of various actions or software. As shown, the manuals GUI 144 allows the user to select and consult an electronic version of the operations manual for the infusion pump 102.

FIG. 8B is a flowchart of a method 700 performed or caused to be performed by execution of the virtual pump manuals software 122 at a processor. As shown, at step 702, the manuals GUI 144 is generated. At step 604, a request for an electronic operations manual is sent to the cloud-based server 180. The requested manual is returned and received at step 706, where a user may review and selected desired data in the manual at step 708. At step 710, inputs for additional actions, if any, are received and the method is repeated as desired.

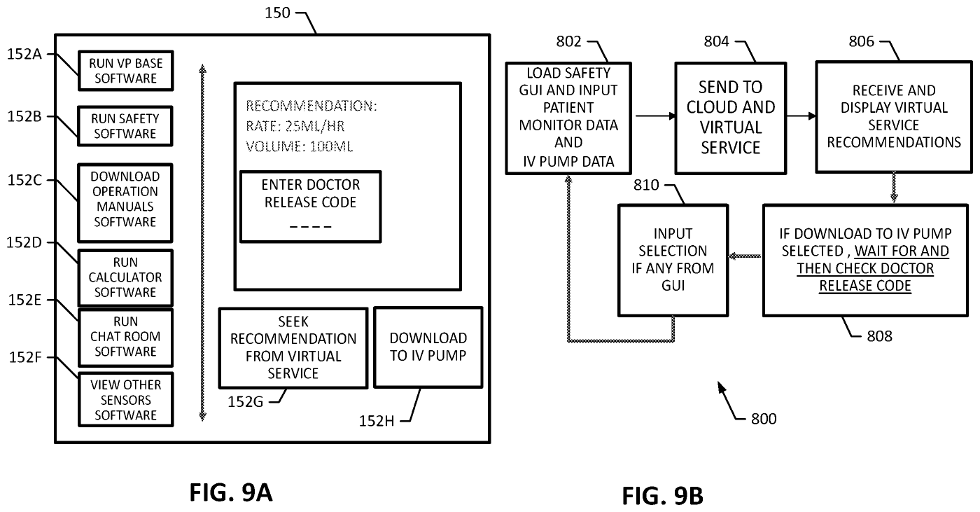

FIGS. 9A-9B are a block diagram of an embodiment of a safety GUI 150 generated by the virtual infusion pump 120 and a flowchart for a method performed by the execution of the safety software 148, respectively. In one embodiment, the safety GUI 150 is generated by the virtual pump 120 for initiating execution of the safety software 148, along with other software executable at the virtual pump. As shown, the safety GUI 150 may also display any security prompts at the virtual pump 120 and includes one or more other interactive input prompts 152A-H to initiate the execution of various actions or software.

FIG. 9B is a flowchart of a method 800 performed or caused to be performed by execution of the virtual pump safety software 148 at a processor. As shown, at step 802, the safety GUI 150 is generated and data from the patient monitor 160 and the infusion pump 102 is received. At step 804, the data from the patient monitor 160 and the infusion pump 102 is sent to the cloud-based server 180. In response, a recommendation returned by the cloud-based server 180 is received and displayed at step 806. If an input to automatically download the recommendations is made the infusion pump 100, then a safety prompt requiring authorization is generated at step 808. If the recommendation is approved and the safety prompt is satisfied at 808, the recommendation is transmitted to the infusion pump 102. At step 810, inputs for additional actions, if any, are received and the method is repeated as desired.

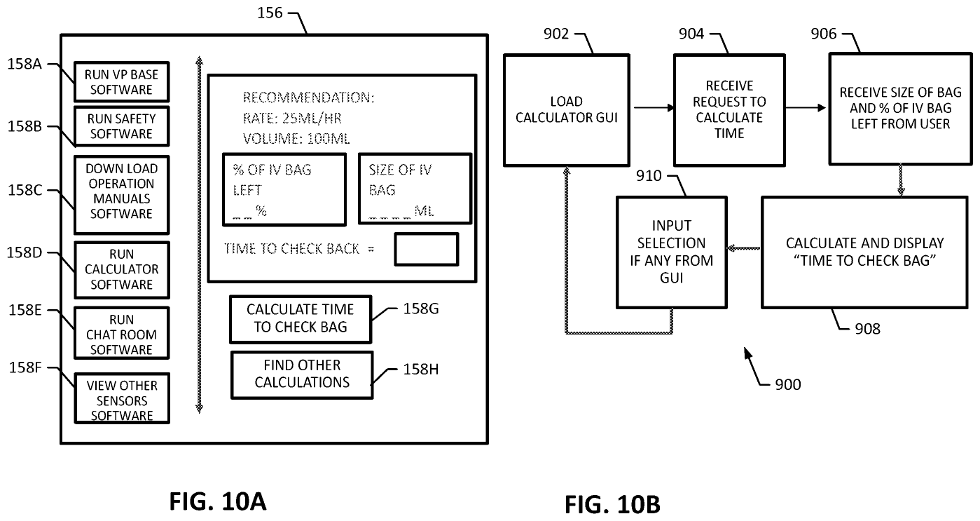

FIGS. 10A-10B are a block diagram of an embodiment of a calculator GUI 156 generated by the virtual infusion pump 120 and a flowchart for a method performed by the execution of the calculator software 154, respectively. In one embodiment, the calculator GUI 156 is generated by the virtual pump 120 for initiating execution of the calculator software 154, along with other software executable at the virtual pump. As shown, the calculator GUI 156 may allow the user to calculate desired values. For example, a user may user the GUI 156 to calculate a time to check an IV bag that is placed in the infusion pump 102. In one aspect, the GUI 156 requests the user to input the size of the IV bag and the percentage of the bag remaining. Using these two values, the total volume of the bag is determined and that is used determine when the bag will run out. In another aspect, the calculator software 154 also relies on flow rate recommendations received by the base software 134 to determine a time when an IV bag will be emptied. The calculator GUI 156 also includes one or more other interactive input prompts 158A-H to initiate the execution of various actions or software.

FIG. 10B is a flowchart of a method 900 performed or caused to be performed by execution of the virtual pump calculator software 154 at a processor. As shown, at step 902, the calculator GUI 156 is generated. At step 904, a request to perform a calculation is received and data for at least one variable of the desired calculation is received at 906. The calculation is performed and the results displayed at the GUI 156 at step 908. At step 910, inputs for additional actions, if any, are received and the method is repeated as desired.

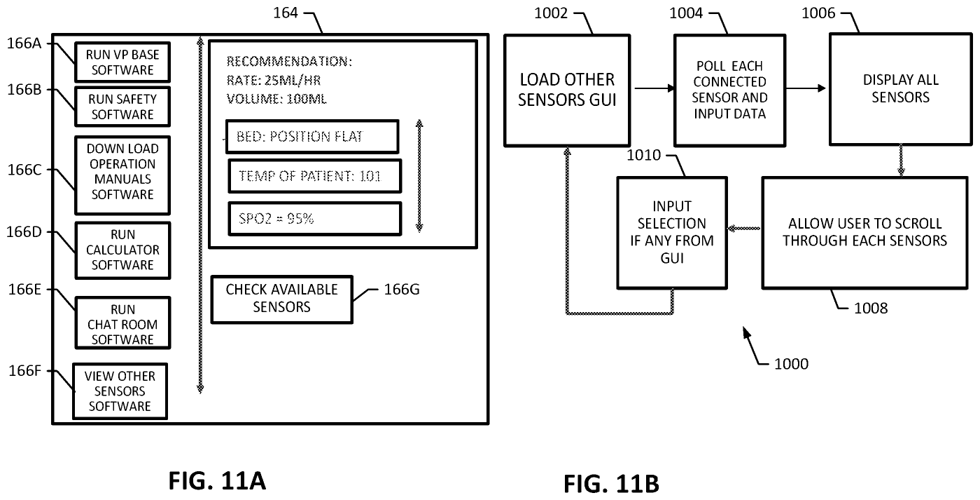

FIGS. 11A-11B are a block diagram of an embodiment of a other sensor GUI 164 generated by the virtual infusion pump 120 and a flowchart for a method performed by the execution of the other sensor software 124, respectively. In one embodiment, the other sensor GUI 164 is generated by the virtual pump 120 for initiating execution of the other sensor software 124, along with other software executable at the virtual pump. As shown, the other sensor GUI 164 allows the user to check data from available sensors and includes one or more other interactive input prompts 156A-G to initiate the execution of various actions or software.

FIG. 11B is a flowchart of a method 1000 performed or caused to be performed by execution of the virtual pump other sensor software 124 at a processor. As shown, at step 1002, the other sensor GUI 164 is generated, while at step 1004, each connected sensor is polled and data, if any, is received. The sensors and data are displayed on the other sensor GUI 164 at 1006 and the user may scroll through the sensors at 1008. At step 1010, inputs for additional actions, if any, are received and the method is repeated as desired.

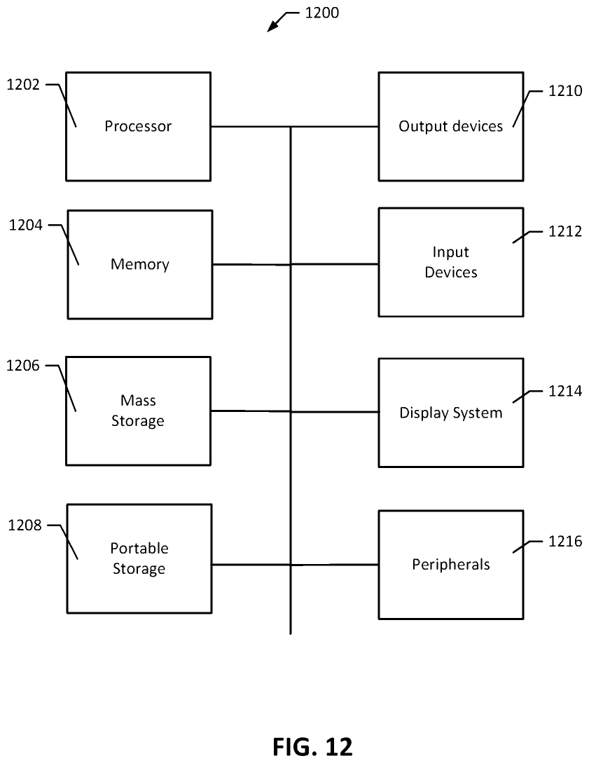

FIG. 12 illustrates an exemplary computing system 1200 that may be used to implement an embodiment of the present invention. The computing system of FIG. 12 includes one or more processors 1202 and memory 1204. Main memory 1204 stores, in part, instructions and data for execution by the processor 1202. Main memory 1204 can store the executable code when in operation. The system of FIG. 12 may further include a mass storage device 1206, portable storage medium drive(s) 1208, output devices 1210, user input devices 1212, a graphics display 1214, and peripheral devices 1216.

The components shown in FIG. 12 are depicted as being connected via a single bus 1218. However, the components may be connected through one or more data transport means. For example, the processor unit 1202 and main memory 1204 may be connected via a local microprocessor bus, and the mass storage device 1206, peripheral device(s) 1216, portable storage device 1208, and display system 1214 may be connected via one or more input/output (I/O) buses.

The mass storage device 1206, which may be implemented with a magnetic disk drive or an optical disk drive, is a non-volatile storage device for storing data and instructions for use by processor unit. The mass storage device 1206 can store the system software for implementing embodiments of the present invention and for purposes of loading that software into main memory 1204.

The portable storage device 1208 operates in conjunction with a portable non-volatile storage medium, such as a floppy disk, compact disk, or Digital video disc, to input and output data and code to and from the computer system of FIG. 12. The system software for implementing embodiments of the present invention may be stored on such a portable medium and input to the computer system via the portable storage device 1208.

Input devices 1212 provide a portion of a user interface. Input devices 1212 may include an alpha-numeric keypad, such as a keyboard, for inputting alpha-numeric and other information, or a pointing device, such as a mouse, a trackball, stylus, or cursor direction keys. Additionally, the system 1200 as shown in FIG. 12 includes output devices 1210. Examples of suitable output devices 1210 include speakers, printers, network interfaces, and monitors.

The display system 1214 may include a liquid crystal display (LCD) or other suitable display device. The display system 1214 receives textual and graphical information, and processes the information for output to the display device.

Peripherals 1216 may include any type of computer support device to add additional functionality to the computer system. For example, peripheral device(s) may include a modem or a router.

The components contained in the computer system 1200 of FIG. 12 are those typically found in computer systems that may be suitable for use with embodiments of the present invention and are intended to represent a broad category of such computer components that are well known in the art. Thus, the computer system 1200 of FIG. 12 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASHEPROM, and any other memory chip or cartridge.

Various forms of transmission media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU. Various forms of storage may likewise be implemented as well as the necessary network interfaces and network topologies to implement the same.

The various computing devices 1200 disclosed herein include computer readable media (CRM) in memory 1204 on which the described applications and software are stored. The computer readable media may include volatile media, nonvolatile media, removable media, non-removable media, and/or another available medium that can be accessed by the processor 1202. By way of example and not limitation, the computer readable media comprises computer storage media and communication media. Computer storage media includes non-transitory storage memory, volatile media, nonvolatile media, removable media, and/or non-removable media implemented in a method or technology for storage of information, such as computer/machine-readable/executable instructions, data structures, program modules, or other data. Communication media may embody computer/machine-readable/executable instructions, data structures, program modules, or other data and include an information delivery media or system, both of which are hardware.

While various flow diagrams provided and described above may show a particular order of operations performed by certain embodiments of the invention, it should be understood that such order is exemplary (e.g., alternative embodiments can perform the operations in a different order, combine certain operations, overlap certain operations, etc.).

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

1. A system for providing a virtual infusion pump, the system comprising:
an infusion pump used in medical treatment of a patient;
one or more sensors that monitor one or more medical factors relevant to a patient, wherein the monitored factors is determined to be associated with a recommended action;
an operation manuals database including an operation manual for the infusion pump; and
a virtual infusion pump controller that formulates instructions for executing the recommended action, wherein the instructions are sent to the infusion pump for execution;
wherein the virtual infusion pump controller requests and receives data from the operation manual.

2. The system of claim 1, further comprising at least one of:
a communication network and a server; wherein the virtual infusion pump controller receives data for the recommended action from the server; and
an experts network, wherein the experts network provides communication between the virtual infusion pump controller and one or more experts.

3. (canceled)

4. The system of claim 1, further comprising operation manuals database, wherein the operation manuals database comprises an operation manual for the infusion pump, and virtual infusion pump controller requests and receives data from the operation manual.

5. The system of claim 1, further comprising at least one of:
a patient monitor, wherein the patient monitor displays data regarding the patient; and
at least one application executable by the virtual infusion pump controller to generate data for modifying the operation of the infusion pump.

6. (canceled)

7. A system for providing a virtual infusion pump, the system comprising:
an infusion pump; and
a virtual infusion pump system comprising:
memory;
a display device;
virtual infusion pump database including an operation manual for the infusion pump; and
a virtual infusion pump processor, the virtual infusion pump processor to:
generate a recommendation graphical user interface;
request and receive data from the operation manual;
receive patient data from at least one of the infusion pump and a patient monitor;
save the received patient data to the virtual infusion pump database;
transmit the patient data and infusion pump data to a cloud-based server, the cloud-based server having a cloud processor, server memory, and at least one cloud database;
receive a recommendation from the cloud-based server, wherein the recommendation is determined by the cloud processor upon the patient data and infusion pump data and accessing the cloud database;
save the recommendation in the virtual infusion pump database;
display the recommendation at the display device; and
transmit the recommendation to the infusion pump.

8. The system of claim 7, further comprising the virtual infusion pump processor to:
generate a chat room graphical user interface;
receive user input at the chat room graphical user interface;
generate a request for an expert;
transmit the request to the cloud-based server;
receive an identification of an available expert;
transmit data to the available expert; and
receive guidance data from the available expert.

9. The system of claim 7, further comprising the virtual infusion pump processor to:
generate an operations manual graphical user interface;
transmit data identifying the infusion pump to the cloud-based server;
receive operations manual data from the cloud-based server; and
display the operations manual data at the display device.

10. The system of claim 7 further comprising the virtual infusion pump processor to:
generate a safety graphical user interface;
generate a safety prompt prior to transmitting the recommendation to the infusion pump; and
receive input to the safety prompt.

11. The system of claim 7, further comprising the virtual infusion pump processor to:
generate a calculations graphical user interface;
receive a user request to perform a calculation;
receive user input for at least one variable of the calculation requested;
determine an answer to the request calculation; and
display the answer at the display device.

12. The system of claim 7, further comprising the virtual infusion pump processor to:
generate an other sensor graphical user interface;
poll a plurality of sensors in communication with the virtual infusion pump system; and,
display a list of sensors proving data to the virtual infusion pump system;

13. A method for providing a virtual infusion pump in a medical network comprising the system of claim 4, the method comprising:
generating a recommendation graphical user interface;
receiving patient data from at least one of the infusion pump and a patient monitor;
saving the received patient data to the virtual infusion pump database;
transmitting the patient data and infusion pump data to a cloud-based server, the cloud-based server having a cloud processor, server memory, and at least one cloud database;
receiving a recommendation from the cloud-based server, wherein the recommendation is determined by the cloud processor upon the patient data and infusion pump data and accessing the cloud database;
saving the recommendation in the virtual infusion pump database;
displaying the recommendation at the display device; and
transmitting the recommendation to the infusion pump.

14. The method of claim 13, further comprising:
at the virtual infusion pump processor:
generating a chat room graphical user interface;
receiving user input at the chat room graphical user interface;
generating a request for an expert;
transmitting the request to the cloud-based server;
receiving an identification of an available expert;
transmitting data to the available expert; and
receiving guidance data from the available expert.

15. The method of claim 13, further comprising:
at the virtual infusion pump processor:
generating an operations manual graphical user interface;
transmitting data identifying the infusion pump to the cloud-based server;
receiving operations manual data from the cloud-based server; and
displaying the operations manual data at the display device.

16. The method of claim 13, further comprising:
at the virtual infusion pump processor:
generating a safety graphical user interface;
generating a safety prompt prior to transmitting the recommendation to the infusion pump; and
receiving input to the safety prompt.

17. The method of claim 13, further comprising:
at the virtual infusion pump processor:
generating a calculations graphical user interface;
receiving a user request to perform a calculation;
receiving user input for at least one variable of the calculation requested;
determining an answer to the request calculation; and
displaying the answer at the display device.

18. The method of claim 13, further comprising:
at the virtual infusion pump processor:
generating an other sensor graphical user interface;
polling a plurality of sensors in communication with the virtual infusion pump system; and
displaying a list of sensors proving data to the virtual infusion pump system.
